(19) United States
(12) Patent Application Publication
(10) Pub. No.: US 2021/0308401 A1
(43) Pub. Date: Oct. 7, 2021

(54) METHOD FOR OPERATING AN ACTUATOR IN A MEDICAL APPARATUS, AND DEVICE THEREFOR

(71) Applicant: IMTMEDICAL AG, BUCHS SG (CH)

(72) Inventor: Matthias VAN DER STAAY, Obstalden (CH)

(21) Appl. No.: 17/266,773

(22) PCT Filed: Aug. 20, 2019
(86) PCT No.: PCT/IB2019/057002

§ 371 Date: Feb. 8, 2021

(30) Foreign Application Priority Data
Aug. 24, 2018 (EP) 18190798.1

(51) Int. Cl. A61M 16/00; A61M 16/06; A61M 16/10; A61M 16/16

(52) U.S. Cl. CPC A61M 16/026; A61M 16/0666; A61M 16/1075; A61M 2016/0039; A61M 2205/3331; A61M 2205/505; A61M 2016/0027; A61M 16/16

(57) ABSTRACT

The invention relates to a method for operating an actuator (22) in a medical apparatus (20), the actuator (22) being connected to a tube system. The medical apparatus (20) comprises: a control apparatus (28) having a pressure controller (35) for controlling a gas pressure; and a computing system (30). The method comprises the following steps: providing a nasal cannula at the tube system; defining a nasal gas end pressure at the pressure controller (35); controlling a nasal gas pressure at the actuator (22) by means of the pressure controller (35), in particular toward the nasal gas end pressure; discharging a conditioned gas from the actuator (22) to the tube system. The invention further relates to a device for operating the actuator (22).

### FIELD OF INVENTION

The invention relates to a method for operating an actuator in a medical apparatus as claimed in claim 1, as well as to a device therefor as claimed in the preamble of claim 9.

### BACKGROUND

Nasal flow therapy or high flow oxygen therapy (HFOT) is a respiratory therapy, in particular for use in hospitals, in which oxygen is supplied to a patient together with compressed air and airway humidification. In this way, flow rates are higher than in conventional oxygen therapy.

High flow oxygen therapy is usually employed with patients who suffer from acute respiratory insufficiency in a clinical setting (for example hypoxaemic respiratory insufficiency). These patients are normally in the intensive care unit or high dependency unit and require support with their breathing in order to stabilise it and so that blood gases can be monitored.

WO 2015/155342 A1 discloses a high flow oxygen therapy system which has a gas supply, a humidifier, a nebulizer, an external nasal interface as well as an aerosol supply line and a gas supply line. The aerosol supply line and the gas supply line are controlled by a valve, wherein a flow control device is provided which controls the flow of aerosol and the flow of gas in the system.

Currently, medical apparatus such as the HFT500 from MEK-ICS CO. LTD. (www.mek-ics.com) is marketed which enables high flow oxygen therapy to be carried out. Medical apparatus of this type comprises an actuator as well as a tubing system connection in order to connect a tubing system to the actuator. The tubing system generally includes a nasal canula.

The disadvantage with known solutions is that the gas flow in medical apparatus of this type is what is adjusted and controlled. The constant gas flow increases the work of breathing, which in turn leads to exhaustion or stress in the patient.

US 2012/0090610 A1 discloses a CPAP apparatus and a method for determining air flow properties of a mask system for the treatment of sleep disorders (SDB), with which different mask systems can be characterized. “Mask system” is used to describe an air delivery hose and a patient connection device or patient interface with a diffuser. The CPAP device has a flow producer which is connected to the mask system and which has a controllable air blower, a flow sensor and a pressure sensor, as well as a processor. The processor is configured in a manner such that air flow properties can be determined at the outlet from the diffuser. Devices of a similar type are disclosed in WO 2011/054038 A1, US 2016/287824 A1, WO 2017/109634 A1 and US 2018/036499 A1.

The disadvantage with the aforementioned solutions is that the devices mentioned therein do not have a pressure controller which is suitable for controlling the nasal gas pressure of different mask systems, whereupon the gas consumption of the device is disproportionately high.

### SUMMARY

The objective of the present invention is to overcome at least partially one or more of the disadvantages of the prior art. In particular, a method for operating an actuator in a medical apparatus and a device therefor will be provided which reduces the gas consumption in the medical apparatus.

The objective is achieved by means of the method and device as defined in the independent claims. Further advantageous embodiments are defined in the figures and in the dependent claims.

The invention concerns a method for operating an actuator in a medical apparatus, wherein the actuator is connected to a tubing system, and the medical apparatus has a control device with a pressure controller for controlling a gas pressure, as well as a computer system, wherein the method comprises the following steps:

providing the tubing system with a nasal cannula (step a));
specifying a final nasal gas pressure in the pressure controller (step b));
adjusting a nasal gas pressure at the actuator with the aid of the pressure controller, in particular towards the final nasal gas pressure (step c)); delivering a conditioned gas from the actuator into the tubing system (step d)).

In particular, the method is carried out before carrying out a nasal flow therapy application or a high flow oxygen therapy on a patient in whom, because of the pressure control during the subsequent nasal flow therapy application, the work of breathing which is expended is lower. In this regard, the pressure controller keeps the gas pressure (which is pre-set) constant. During an application, the peak flow demands on the gas or the respiratory gas can be covered, and the gas consumption in the medical apparatus is reduced, wherein the applied oxygen concentration remains unchanged. The nasal gas pressure is the gas pressure pertaining to the tubing system. A conditioned gas has a gas pressure and gas flow which is predetermined by the actuator. The tubing system generally comprises a tube and a nasal canula.

Advantageously, the medical apparatus is a ventilator wherein, with the aid of the pressure control at the actuator, the pressure in the lungs of the patient can be controlled better or can be adjusted better. With the method in accordance with the invention, the patient experiences enhanced comfort because of an adaptive gas flow.

Preferably, the pressure control is carried out on the basis of at least one measured nasal pressure value or on the basis of at least one pressure approximation. In the case of pressure control with at least one measured nasal pressure value, the method described here can be carried out without an adjustment step. Here, the medical apparatus adapts itself automatically to changes in the tubing system. In the case of a control of pressure on the basis of at least one pressure approximation, a nasal pressure measurement can be delivered with, whereupon the tubing system can be produced more cheaply and the medical device can be operated more universally with different tubing systems or tubing system accessories.

Particularly preferably, the nasal gas flow in the tubing system varies. In this manner, the at least one pressure approximation can be determined for different nasal gas flows, during which the nasal canula is typically not connected to a patient. The variation in the nasal gas flow may be linear, for example, so the at least one pressure approximation can easily be determined. The nasal gas flow is the gas flow in the tubing system.

Preferably, the variation in the nasal gas flow is between 0 litres per minute and 100 litres per minute. In this regard, the variation in the nasal gas flow may be from high gas flow values to low gas flow values or vice versa, from low gas flow values to high gas flow values. In this manner, a plurality of pressure approximations can be determined which may act as the basis for a particularly accurate pressure control at the actuator.

In particular, the variation in the nasal gas flow occurs in a previously determined time interval, for example within 10 seconds. In this manner, the at least one pressure approximation can be determined in a reproducible manner.

Preferably, the variation in the nasal gas flow in the tubing system occurs before setting the final nasal gas pressure at the pressure controller (step b)). In this manner, before being used on the patient, the medical apparatus can be adjusted to the apparatus-tubing system configuration, whereupon gas pressures that are too high can be avoided and subsequently, enhanced patient safety is ensured.

Preferably, the at least one pressure approximation is calculated on the basis of at least one internal measured pressure value and on the basis of at least one differential pressure approximation in the tubing system. In this regard, the at least one internal measured pressure value may be determined in the medical apparatus with a pressure measuring sensor which is disposed in the region of the tubing system connection. In this manner, a pressure measuring sensor which is external to the tubing system is not necessary.

Preferably, the at least one differential pressure approximation in the tubing system is calculated on the basis of the internal gas flow. The internal gas flow is measured with the aid of a flow measuring sensor disposed in the medical apparatus or is measured indirectly via a suitable differential pressure-based flow measurement method. In this manner, the at least one pressure approximation can be accurately determined with the aid of a simple measurement setup with precisely one pressure measuring sensor and with precisely one flow measuring sensor, whereupon the gas consumption in the medical apparatus is determined exactly and too much subsequent gas consumption is avoided.

In particular, the at least one differential pressure approximation is calculated with the aid of a mathematical function, whereupon the at least one pressure approximation can be reliably determined in one calibration step. As an example, the mathematical function is a polynomial function or a quadratic function which is solved with a least square method, for example. In this manner, the at least one pressure approximation is determined particularly easily.

Preferably, the at least one differential pressure approximation is called up from a table in the computer system. In this regard, the at least one differential pressure approximation can be provided or pre-configured by the manufacturer of the medical apparatus or stored in the table of the computer system during the calibration steps. In this regard, the differential pressure approximations are associated with the respective internal gas flow values, so that a differential pressure approximation can be deduced from a measured gas flow value.

In particular, the at least one differential pressure approximation is determined with the aid of at least one measured nasal pressure value. The at least one differential pressure approximation can also be determined from it during the use of the medical apparatus, whereupon patient safety is enhanced. In this regard, the at least one internal measured pressure value is supplemented with at least one measured nasal pressure value and the at least one pressure approximation is determined from this. In this manner, the nasal pressure measurement is used in order to automatically recognise a variation in the tubing system—for example after changing the tubing system with a nasal cannula.

Preferably, a minimum gas flow through the tubing system is determined. By applying a minimum gas flow, carbon dioxide clearance in respiratory gas can be ensured. In this regard, the minimum gas flow may simply be entered into the medical apparatus by the user on the basis of the height or ideal body weight and/or gender of a patient, or with the aid of established theories, for example based on OTIS et al.

Preferably, the minimum gas flow is calculated in the computer system using:

$${F}_{min} = \frac{{V}_{d}}{k * {T}_{e}}$$

wherein V_{d} is the dead space, k is a factor between 0.05 and 2, and Tₑ is an expiration time. The minimum gas flow can therefore be specified without the aid of a user of the medical apparatus, reducing their load.

In particular, the dead space V_{d} is calculated with the aid of an ideal body weight of the patient and a Radford constant, wherein the Radford constant is typically 2.2 mL/kg. This means that the dead space V_{d} can easily be determined for a majority of applications.

Preferably, the factor k=0.33 is used for the calculation of the minimum gas flow, whereupon the minimum gas flow can easily be calculated and particularly good patient safety is guaranteed.

In particular, Tₑ is an expiration time which is calculated with the computer system. In this manner, the minimum gas flow can be matched to the current respiratory activity of the patient.

Alternatively, the minimum gas flow is determined with the aid of a flow approximation which is determined on the basis of an internal gas flow and on the basis of a leakage flow in the tubing system. In this manner, the leakage flow can typically be calculated with the aid of the nasal gas pressure.

In this regard, the dead space V_{d} is estimated from the estimated values derived from the flow approximation, for example on the basis of a volume per minute Mᵥ, a respiratory frequency f, an expiration time constant RC, so that the determination of the dead space V_{d} is carried out automatically in the computer system of the medical apparatus. As an example, the dead space V_{d} is determined using:

$${V}_{d} = \frac{{M}_{v}}{( 2 * f + {π}^{2} R C * {f}^{2} )}$$

Next, the minimum gas flow can be calculated using this dead space V_{d}, as described above. In this regard, the user does not need to make any adjustments to the medical apparatus. The algorithm ensures that the carbon dioxide clearance always lies in the optimum range.

Preferably, the minimum gas flow is set to a value between 0 and 100 litres per minute. In this manner, the minimum gas flow can be limited to a value that is suitable for a patient.

Preferably, the previously calculated minimum gas flow is limited, wherein for this, an effective, internal, maximum gas flow is used as the starting point. The effective, internal, maximum gas flow of the gas in the tubing system can be determined in the medical apparatus, for example by measuring the gas flow with an internal flow measuring sensor in the medical apparatus over a specified period of time. Typically, this period of time is in the range between 10 seconds and 60 seconds, in particular in the range between 25 seconds and 45 seconds, in particular 36 seconds. By limiting the internal gas flow further, carbon dioxide clearance which is calculated in situ is ensured in the gas.

In particular, the calculated minimum gas flow is limited with an upper boundary of 0.8 times the effective, internal, maximum gas flow and with a lower boundary of 0.2 times the effective, internal, maximum gas flow. In this manner, patient safety is further improved.

Preferably, a maximum, mean gas flow is established through the tubing system. In this regard, it is permissible for the gas flow to periodically exceed a maximum, but on average to remain below the mean maximum gas flow, wherein the maximum should be understood to be the limit. The limit guarantees sufficient humidification of the gas which is provided in the tubing system.

Preferably, the mean, maximum gas flow is set to a value between 10 litres per minute and 200 litres per minute. This ensures that the patients can be supplied with sufficiently humidified gas.

Particularly preferably, the mean, maximum gas flow is set to a value of 60 litres per minute. In this manner, a sufficiently humidified gas is provided for the majority of applications, so that the user does not have to worry about explicitly setting this parameter on the medical apparatus.

In particular, the mean, maximum gas flow is regulated on the basis of a measured gas humidity value. In this regard, with the aid of the specific measured gas humidity value, the mean, maximum gas flow is controlled by the flow controller in the medical apparatus. In this manner, an optimally humidified gas is provided in the tubing system. As an example, the medical apparatus is connected to a humidifier or itself comprises a humidifier.

As an alternative, the mean, maximum gas flow is determined on the basis of an oxygen concentration in the gas. In this regard, the oxygen concentration is typically measured or determined. In this manner, a high mean oxygen flow is prevented.

Preferably, the nasal gas flow is controlled in a manner such that it is always in the range between the minimum gas flow and the mean, maximum gas flow, and thus carbon dioxide clearance in patients and humidification of the gas are ensured and excessively high oxygen consumption is dealt with.

Preferably, the nasal gas flow is controlled as an inner cascade for the nasal gas pressure, wherein a cascaded control structure is used which ensures a nasal gas flow between the required minimum gas flow and the required mean, maximum gas flow.

Preferably, setting of the final nasal gas pressure (step b)) is carried out automatically, so that the user of the medical apparatus does not have to set this parameter manually, thus providing enhanced patient safety. In this regard, the control algorithm in the computer system of the medical apparatus takes over specification of the final nasal gas pressure.

Preferably, the control algorithm carries out a zero adjustment from a minimum, internal gas flow measured in the medical apparatus and the minimum gas flow and adjusts the nasal gas pressure towards the final nasal gas pressure. To express this another way, the control algorithm raises the nasal gas pressure when the measured minimum, internal gas flow is smaller than the minimum gas flow to be reached which is specified by the medical apparatus or by the user of the medical apparatus, or the control algorithm reduces the nasal gas pressure when the measured minimum internal gas flow is higher than the minimum gas flow to be reached as specified by the medical apparatus or by the user of the medical apparatus, wherein the inner cascaded flow controller does not limit the minimum gas flow. In this manner, the medical apparatus always adjusts towards an optimal final nasal gas pressure.

The minimum internal gas flow of the gas in the medical apparatus can be determined by measuring this gas flow with an internal flow measuring sensor in the medical apparatus over a specified period of time. Typically, this period of time is in the range between 0.5 seconds and 50 seconds, in particular in the range between 2 seconds and 30 seconds.

Preferably, the final nasal gas pressure is adjusted on the basis of a blood gas value. As an example, the blood gas value is measured in advance. The final nasal gas pressure is raised or lowered as a function of the value of the measured blood gas value. In this manner, the final nasal gas pressure can be set for each individual patient.

In particular, the blood gas value is a carbon dioxide value—for example a transcutaneous carbon dioxide value or an arterial carbon dioxide partial pressure- or an oxygen saturation value—for example a blood oxygen saturation value. In this manner, current blood gas values can be used in order to adjust the final nasal gas pressure.

Preferably, the final nasal gas pressure is limited to a value between 0.1 mbar and 20 mbar, so that the gas consumption can be kept low.

Preferably, on the basis of the previously determined nasal gas flow, the control device generates a suitable control command for the actuator, so that the actuator appropriately delivers the conditioned gas into the tubing system. In this manner, the gas consumption in the medical apparatus is optimized. The control command is a voltage, a rotational speed value or a current value or is provided on another basis, although this list is not limiting.

Alternatively or in addition, on the basis of the previously determined nasal gas pressure, the control device generates a suitable control command for the actuator, so that the actuator appropriately delivers the conditioned gas into the tubing system. In this manner, not only is the gas consumption in the medical apparatus optimized, but also enhanced patient comfort is ensured.

In a further aspect, the invention concerns a device for carrying out a nasal flow therapy application, comprising an actuator, a tubing system connection for connecting a tubing system to the actuator as well as at least one transducer which provides measuring signals from at least one pressure measuring sensor. Furthermore, a control device with a pressure controller for controlling a nasal gas pressure is provided.

The pressure controller in the device, for example in a medical apparatus, enables a patient to expend less actual work of breathing during a nasal flow therapy application. In this regard, the pressure controller is configured such that it is regulated to a constant gas pressure. During an application, peak demands for the gas or the respiratory gas can be covered and a lower gas consumption can be obtained in the medical apparatus, whereupon the applied oxygen concentration can remain unchanged.

Typically, an actuator described here comprises at least one blower for conditioning the gas, whereupon the medical apparatus can be used in a private household.

Alternatively or in addition, an actuator as described here comprises at least one source of gas with a valve for conditioning the gas. In this manner, the infrastructure present in a hospital may be used. As an alternative, when using a blower and a gas source with a valve, the infrastructure in the hospital can support the medical apparatus. Furthermore, the different oxygen concentrations brought about by this can be specified directly on the medical apparatus.

Preferably, a computer system is provided, wherein the actuator is connected to a computer system and the computer system is configured to calculate at least one pressure approximation on the basis of at least one internal measured pressure value and on the basis of at least one differential pressure approximation in the tubing system. In this regard, the at least one internal measured pressure value can be determined with a pressure measuring sensor which is disposed in the region of the tubing system connection. In this manner, no additional pressure measuring sensors are required in the tubing system. Typically, a digital-to-analogue transducer is located in the device—preferably between the computer system and the actuator—wherein the digital-to-analogue transducer converts the values calculated in the computer system into control commands for the actuator.

In particular, the computer system has a computer algorithm which is configured to execute the method described here. In this manner, the method described here can be implemented entirely automatically.

Preferably, the computer system has a storage unit. In this manner, the values which can be used for the pressure control, such as the at least one internal measured pressure value or the at least one differential pressure approximation, can be stored in the storage unit so that they can easily be called up when needed.

Preferably, the pressure controller is configured to control the nasal gas pressure on the basis of the at least one pressure approximation. When pressure control is carried out on the basis of at least one pressure approximation, a nasal pressure measurement can be delivered with, whereupon the nasal cannulas can be produced more cheaply and the medical apparatus can be operated with different tubing systems or tubing system accessories and thus be operated in a more universal manner.

More preferably, a pressure measuring device is provided for recording at least one measured nasal pressure value, wherein the pressure controller controls the nasal gas pressure pertaining to the tubing system on the basis of the at least one measured nasal pressure value. In this manner, the nasal pressure measurement is used in order to automatically detect a variation in the tubing system—for example a change of nasal canula in the tubing system—whereupon a calibration step can be delivered with.

Preferably, a flow measuring sensor is provided in the medical apparatus for measuring an internal gas flow. The flow measuring sensor preferably measures the internal gas flow delivered by the actuator, whereupon the gas consumption can be optimized and carbon dioxide clearance can be ensured.

Preferably, a humidifier is provided, wherein the humidifier is preferably disposed on the tubing system. Humidified high flow oxygen therapy is successfully used with patients with COPD, bronchiectasis, end stage cancer and non-intubated patients who require optimally humidified gas.

In particular, the humidifier is configured to set the temperature of the gas. In this manner, the humidification in the gas can be further optimized.

Preferably, a temperature control system for controlling the temperature of the conditioned gas is provided, whereupon condensation of the humidified air is prevented and the partial pressure of the water vapour which reaches the patient is raised in this manner.

Preferably, the temperature control system for controlling the temperature of the conditioned gas is disposed in the tubing system, whereupon the temperature control is customized for the tubing system and optimally temperature-controlled gas is provided.

Alternatively, a humidifier as well as a temperature control system are present, whereupon an optimally conditioned gas is provided. Preferably, the humidifier and the temperature control system are combined into one system.

Alternatively, the humidifier and/or the temperature control system is/are disposed in the device. The size of a humidifier built into the device is matched to the parameters of the device so that the device can be manufactured inexpensively and the user does not need additional appliances.

More preferably, a flow controller for controlling a nasal gas flow is provided. In this manner, in addition to controlling the pressure, the flow is also controlled, by means of which the gas consumption, carbon dioxide clearance and humidification can be ensured.

In particular, a cascaded control structure is provided, wherein the pressure controller for controlling the nasal gas pressure forms an outer cascade and the flow controller for controlling a nasal gas flow forms an inner cascade. In this manner, the gas flow through the tubing system described herein cannot go below the minimum, or in contrast, the maximum mean gas flow through the tubing system described herein cannot be exceeded, whereupon as a consequence, clearance of the carbon dioxide content in the gas, sufficient humidification of the gas as well as optimal gas consumption are ensured.

Preferably, an oxygen dosing device is provided, whereupon an oxygen concentration can be specified with the device.

Preferably, an input device is provided, so that the user of the device can designate settings such as, for example, the final nasal gas pressure and/or the oxygen concentration manually or can input them using it.

In particular, the input device has a display unit. In this regard, typically, the settings designated by the user are displayed, whereupon the user can readily visually check the settings on the device.

Preferably, the input device is a touch screen, and so the device is easy to operate and has a particularly compact input device.

Preferably, the input device has at least one switch device for selecting between automatic pressure control or constant pressure control. In the case of automatic pressure control, the user themselves cannot set a final nasal gas pressure, but rather, the medical apparatus autonomously calculates the optimal final nasal gas pressure. The switch device is integrated into the touch screen. In this manner, the inputs can easily be entered by the operator of the medical apparatus. As an alternative, the switch device is configured as a mechanical switch. In this way, the operator could, for example, be able to see the position of the mechanical switch from a distance.

More preferably, a measuring device for blood gas measurement is provided. The final nasal gas pressure can be adjusted automatically on the basis of the measured blood gas values extracted from this.

Preferably, a carbon dioxide measurement is provided. In this regard, the carbon dioxide measured values are taken into consideration during pressure control, so that a sufficiently high gas pressure as well as a sufficiently high gas flow are provided.

Alternatively or in addition, oxygen measurement is provided. The oxygen measurement is connected to the device or is integrated into the device. In this regard, the measured oxygen values are taken into consideration, so that a sufficient quantity of oxygen is provided.

Preferably, the method described here is a computer-implemented method for operating an actuator. The control device and the computer system in the medical apparatus are configured in this regard in a manner such that the method described here is executed automatically. In this manner, the production costs are reduced.

Preferably, the computer-implemented method disclosed here is stored in a storage medium. The storage medium can on the one hand be integrated into the device or on the other hand be a mobile storage medium. A mobile storage medium may optionally be connected to different devices so that the method disclosed here can be carried out at different locations.

In particular, control commands for the actuator are stored in the storage medium. The storage medium is inserted into the medical apparatus so that they can be accessed immediately and the actuator can be operated with these control commands.

Further advantages, features and details of the invention will become apparent from the following description in which exemplary embodiments of the invention are described with reference to the drawings.

The list of reference numerals as well as the technical content of the patient claims and figures form part of the disclosure. The figures have been described clearly and comprehensively. Identical reference numerals identify identical components; reference numerals with different indices indicate components with identical or similar functions.

### BRIEF DESCRIPTION OF THE DRAWINGS

In the figures:

FIG. 1 shows a system for carrying out a nasal flow therapy application,

FIG. 2 shows a first embodiment of a device in accordance with the invention for carrying out a nasal flow therapy application,

FIG. 3 shows a further embodiment of a device in accordance with the invention for carrying out a nasal flow therapy application,

FIG. 4 shows a flow diagram for a first embodiment of the method in accordance with the invention, without a pressure measuring device, and

FIG. 5 shows a flow diagram for a further embodiment of the method in accordance with the invention, with a pressure measuring device.

### DETAILED DESCRIPTION OF THE PREFERRED EMBODIMENT

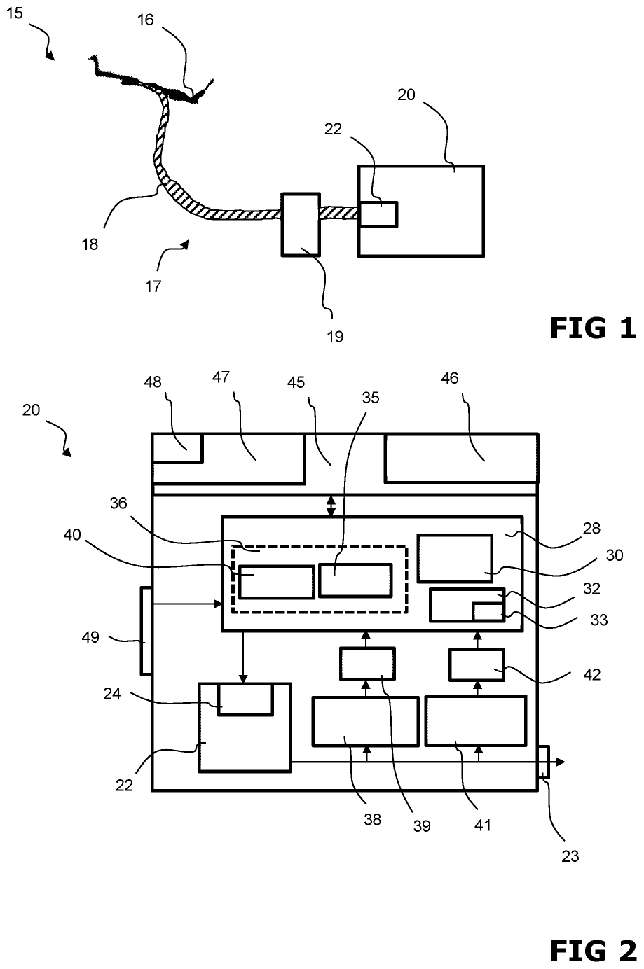

FIG. 1 shows a system 15 for carrying out nasal flow therapy on a patient. The system 15 comprises a tubing system 17 and a medical apparatus 20 with an actuator 22. The tubing system 17 comprises a tube 18 and a nasal canula 16, and is connected to the actuator 22 of the medical apparatus 20. A humidifier 19 is disposed on the tubing 18. The actuator 22 makes conditioned gas available to the tubing system 17 or the nasal canula 16 and which is delivered to the environment or to a patient through the tubing system 17 or through the nasal canula 16. Depending on the composition of the gas, this could be a respiratory gas, for example, which is administered to a patient to support breathing.

FIG. 2 shows a first embodiment in accordance with the invention of the medical apparatus 20 from FIG. 1 and comprises a tubing system connection 23 for connecting up the tubing system 17. The actuator 22 in the medical apparatus 20 has a digital-to-analogue transducer 24 and is connected to the tubing system connection 23. In a non-limiting manner, the actuator 22 consists of a blower and/or an oxygen connection and a valve and in particular a humidifier (not shown).

The medical apparatus 20 has a control device 28 which contains a computer system 30, a storage unit 32 and a cascaded control structure 36. In the control device 28, the computer system 30, the storage unit 32 and the cascaded control structure 36 are connected together in order to exchange data. The storage unit 32 has a table 33 for storing gas pressure data and gas flow data. The control device 28 has a pressure controller 35 for controlling a nasal gas pressure Pₙₐₛₐₗ, which is connected to the flow controller 40 in order to exchange data. The pressure controller 35 is configured such that the actuator 22 keeps the gas at a constant nasal gas pressure Pₙₐₛₐₗ. The control device 28 has a flow controller 40 for controlling the nasal gas flow Fₙₐₛₐₗ, which is electrically connected to the digital-to-analogue transducer 24 of the actuator 22. In this regard, the pressure controller 35 and the flow controller 40 are connected together by means of a cascaded control structure 36, wherein the pressure controller 35 forms an outer cascade and the flow controller 40 forms an inner cascade.

The medical apparatus 20 has an internal pressure measuring sensor 38 for measuring internal gas pressures Pᵢₙₜ which are specified or produced by the actuator 22. These are provided to the control device 28 as pressure measuring signals with the aid of an analogue-to-digital transducer 39.

The medical apparatus 20 has a flow measuring sensor 41 for measuring internal gas flows Fᵢₙₜ, which are specified or produced by the actuator 22. These are provided to the control device 28 as flow measuring signals with the aid of an analogue-to-digital transducer 42.

An input and output device 45 is disposed on the medical apparatus 20 and provides a display unit 46 with a touch screen 47 for inputting and displaying gas pressure values and/or gas flow values or gas pressure data and/or gas flow data. The input and output device 45 furthermore comprises a switch device 48 for selecting between an automatic pressure control and a constant pressure control. The input and output device 45 is connected to the control device 28 in order to exchange data.

The medical apparatus 20 has a terminal strip 49 for connecting measuring devices or dosing devices. The terminal strip 49 is connected to the control device 28. Non-limiting examples in this regard which may be connected are a measuring device for measuring blood gas such as a carbon dioxide measurement and/or an oxygen measurement, an oxygen dosing device with an oxygen connection and a valve (not shown).

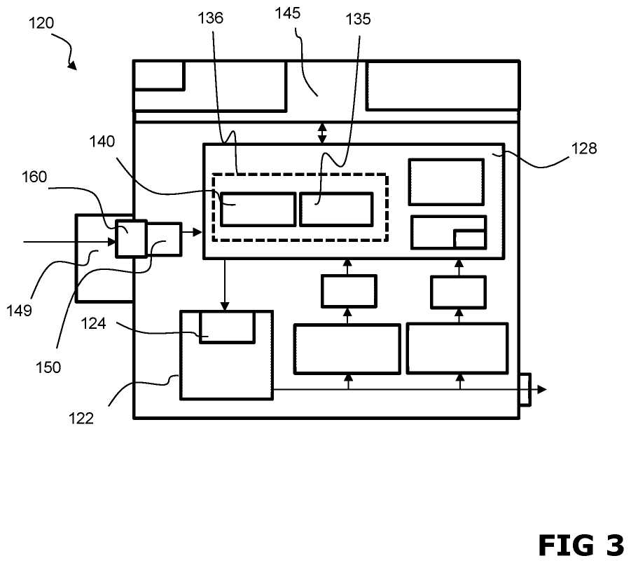

The medical apparatus 120 shown in FIG. 3 substantially corresponds to the medical apparatus 20 as described in relation to FIG. 2. The medical device 120 differs therefrom in that a pressure measuring device 160 for detecting measured nasal pressure values Pₘₑₛ is provided and the medical apparatus 120 is preferably a ventilator. In this regard, the medical apparatus 120 has a control device 128 which has the components and their technical functions described in respect of FIG. 2. Furthermore, the medical device 120 has an input and output device 145 which has the components and their technical functions described in respect of FIG. 2.

The pressure measuring device 160 is connected to the terminal strip 149. The measured nasal pressure values Pₘₑₛ measured by the pressure measuring device 160 are transmitted to the control device 128 with the aid of the analogue-to-digital transducer 150. The measured nasal pressure values Pₘₑₛ are converted into control commands in the pressure controller 135 of the control device 128 in order to control the actuator 122. The control device 128 has a flow controller 140 for controlling the nasal gas flow Fₙₐₛₐₗ, which is electrically connected to the digital-to-analogue transducer 124 of the actuator 122. In this regard, the pressure controller 135 and the flow controller 140 are connected together by means of a cascaded control structure 136, wherein the pressure controller 135 forms an outer cascade and the flow controller 140 forms an inner cascade. The control commands are transferred to the actuator 122 by means of the digital-to-analogue transducer 124.

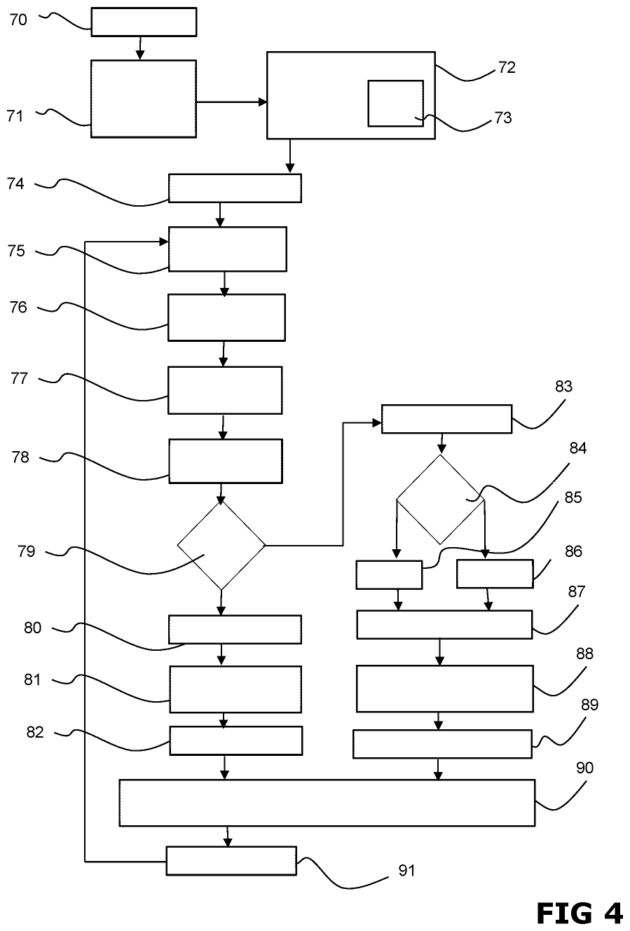

FIG. 4 shows a first embodiment of the method in accordance with the invention for operating the actuator 22 described above in a medical apparatus 20 in accordance with FIG. 1 and FIG. 2. The tubing system connection 23 has already been connected to the tubing 18 of the tubing system 17 and the nasal canula 16 has been provided on or positioned on the tubing system 17 (step 70; step a)). Next, it is ensured that the nasal canula 16 has not been placed on a patient (step 71). Then follow several steps for calibrating the medical apparatus 20 together with the tubing system 17, wherein the internal gas flow Fᵢₙₜ of the gas is raised in a linear manner from 0 litres per minute to 100 litres per minute within a period of time of 10 seconds (step 72). During this period of time, the internal gas pressure values Pᵢₙₜ and the internal gas flow values Fᵢₙₜ are measured with the internal pressure measuring sensor 38 and the internal flow measuring sensor 41 and transferred to the control device 28. The respective differential pressure approximations dP_{sch} are then calculated on the basis of the measured internal gas pressure or the measured internal gas pressure value Pᵢₙₜ and on the basis of the pressure approximation Pₙ in the computer system 30, wherein:

Pₙ=Pᵢₙₜ−dP_{sch},

and when Pₙ=0, then dP_{sch}=Pᵢₙₜ.

In this manner, the several internal gas measured pressure values Pᵢₙₜ correspond to the differential pressure approximations dP_{sch} in the tubing system 17. These are stored in the table 33 of the storage unit 32 together with the measured internal gas flow values Fᵢₙₜ associated with them (step 73), whereupon the medical apparatus 20 has been calibrated for the tubing system connected to it.

Alternatively, the respective differential pressure approximations dP_{sch} can be calibrated using mathematical functions, for example polynomial functions such as:

dP_{sch}=R₀+R₁*Fᵢₙₜ+R₁*Fᵢₙₜ²+ . . .

The constants R₀, R₁, . . . are then determined with the aid of a least square method. Further functions for determining the differential pressure approximation dP_{sch} on the basis of the internal gas flow Fᵢₙₜ are linear functions or quadratic functions; this list is not exhaustive.

In a further step (step 74), the maximum, mean gas flow Fₘₐₓ through the tubing system 17 is specified to be 100 litres per minute.

Next, an ideal body weight for a patient (IBW) (or the height and gender of the patient) and an oxygen concentration FiO₂ in the medical apparatus 20 is set on the input and output device 45 and a measured, effective, internal, maximum gas flow F_{int, max} and an expiration time Tₑ are calculated in the computer system 30 (step 75).

Furthermore, the dead space V_{d} is calculated with the aid of the ideal body weight (IBW) and a Radford constant, wherein the Radford constant is typically 2.2 mL/kg (V_{d}=2.2 mL/kg×IBW) (step 76).

Next, the respective pressure approximation Pₙ is determined with the aid of the measured internal gas pressure values Pᵢₙₜ and the differential pressure approximations dP_{sch} stored in the table 33, whereupon the differential pressure approximation dP_{sch} is determined with the aid of the measured internal gas flow Fᵢₙₜ (step 77).

Subsequently, a minimum gas flow Fₘᵢₙ through the tubing system is determined (step 78).

In this regard, the minimum gas flow Fₘᵢₙ is calculated in the computer system 30 using:

$${F}_{min} = \frac{{V}_{d}}{k * {T}_{e}}$$

wherein V_{d} is the previously determined dead space, k is 0.33 and Tₑ is the expiration time which has already been calculated.

In a further step (step 79), the medical apparatus 20 is interrogated as to whether the final nasal gas pressure P_{nSet} is to be set automatically. The interrogation is made by the position of the switch device 48 on the medical apparatus 20 or by means of a specified setting in the control device 28.

In the case of non-automatic setting of the final nasal gas pressure P_{nSet}, a set final nasal gas pressure P_{nSet} is manually entered by the user with the input and output device 45 on the medical apparatus 20 (step 80; step b)).

Next, the final gas flow F_{nSet} is determined as a function of the difference between the set final nasal gas pressure P_{nSet} and the previously determined pressure approximations Pₙ, so that the difference mentioned above tends towards 0 (zero adjustment), according to:

Calculate F_{nSet} so that: P_{nSet}−Pₙ→0

whereupon the nasal gas pressure Pₙₐₛₐₗ is adjusted towards the final nasal gas pressure P_{nSet} (step 81; step c)). In this regard, the pressure control 35 is carried out first as the outer cascade of the cascaded control structure.

Furthermore, the upper limit of the final gas flow F_{nSet} determined in step 81 is the previously determined maximum, mean gas flow Fₘₐₓ and the lower limit is the previously determined minimum gas flow Fₘᵢₙ (step 82).

As an alternative to the steps mentioned immediately above (steps 80 to 82), in the case of an automatic determination of the final nasal gas pressure P_{nSet}, a determination of a measured, effective, internal, minimum gas flow F_{int, min} is carried out (step 83) as well as an interrogation as to whether the calculated minimum gas flow Fₘᵢₙ is smaller than the determined measured, effective, internal, minimum gas flow F_{int,min} (step 84).

If the calculated minimum fragment Fₘᵢₙ is smaller than the determined measured, effective, internal, minimum gas flow F_{int,min}, the control device 28 reduces the final nasal gas pressure P_{nSet} and sets it (step 85; step b)).

If the calculated minimum fragment Fₘᵢₙ is greater than the determined measured, effective, internal, minimum gas flow F_{int,min}, the control device 28 increases the final nasal gas pressure P_{nSet} and sets it (step 86; step b)).

In both cases, the final nasal gas pressure P_{nSet} is limited to a value between 0 mbar and 10 mbar (step 87).

Next, in step 88, the pressure control is carried out as the outer cascade of the cascaded control structure.

The upper limit of the final gas flow F_{nSet} is the previously determined maximum, mean gas flow Fₘₐₓ and the lower limit is the value zero (step 89).

Next, the measured, internal gas flow Fᵢₙₜ is adjusted towards the calculated final gas flow F_{nSet} with the aid of the flow controller 40 and subsequently with the aid of the actuator 22 (step 90). In other words, a zero adjustment is carried out, in accordance with

F_{nSet}−Fᵢₙₜ→0

so that the nasal gas flow Fₙₐₛₐₗ is adjusted towards the calculated final gas flow F_{nSet}. In this manner, the flow control is carried out as the inner cascade of the cascaded control structure.

Next, the control device 28 generates a suitable control command for the actuator 22 on the basis of step 90 and transfers it to the actuator 22, so that the actuator 22 delivers the conditioned gas into the tubing system (step 91; step d)).

Subsequently, steps 75 to 82 and step 89 to step 91 can be carried out several times.

As an alternative to this, the steps 75 to 79 and the steps 83 to 91 may be carried out several times.

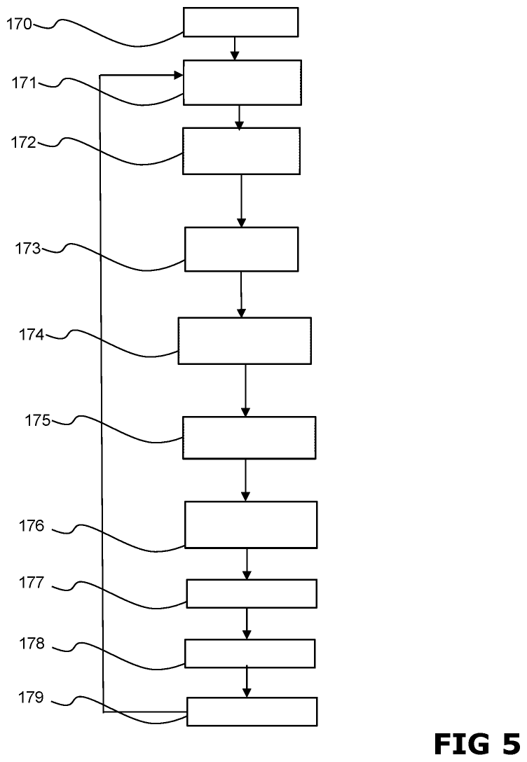

FIG. 5 shows a further embodiment of the method in accordance with the invention for operating the actuator 122 described above in a medical apparatus 20 in accordance with FIG. 1 and in a medical apparatus 120 in accordance with FIG. 3, wherein the medical apparatus 120 comprises a pressure measuring device 160 or is connected to it, and which is configured to detect measured pressure values Pₘₑₛ.

In a first step (step 170), the maximum mean gas flow Fₘₐₓ through the tubing system is set to 100 litres per minute.

Next, an ideal body weight of a patient (IBW) (or the height and gender of the patient) and an oxygen concentration (FiO₂) is designated in the medical apparatus 120, and a measured, effective, internal, maximum gas flow F_{int,max} and an expiration time Tₑ are calculated (step 171).

Furthermore, the dead space V_{d} is calculated with the aid of the ideal body weight (IBW) and a Radford constant, wherein the Radford constant is typically 2.2 mL/kg (V_{d}=2.2 mL/kg×IBW) (step 172).

Subsequently, a minimum gas flow Fₘᵢₙ through the tubing system is determined (step 173).

In this regard, the minimum gas flow Fₘᵢₙ is calculated in the computer system using:

$${F}_{min} = \frac{{V}_{d}}{k * {T}_{e}}$$

wherein V_{d} is the previously determined dead space, k is 0.33 and Tₑ is the expiration time which has already been calculated.

Next, a final nasal gas pressure P_{nSet} is manually input into the medical apparatus 120 by the user using the input and output device 145 (step 174; b)).

Subsequently, the measured nasal pressure value (Pₘₑₛ) is measured with the pressure measuring device 160 and transferred to the pressure controller 135 of the control device 128 via the analogue-to-digital transducer 150 (step 175).

Next, the final gas flow F_{nSet} is determined as a function of the difference between the set first final nasal gas pressure P_{nSet} and the previously measured nasal pressure value Pₘₑₛ, so that the difference mentioned above tends towards 0 (zero adjustment), according to:

Calculate F_{nSet} so that: P_{nSet}−Pₘₑₛ→0

whereupon the nasal gas pressure Pₘₑₛ is adjusted towards the first final nasal gas pressure P_{nSet} (step 176; step c)). In this regard, the pressure control is carried out first as the outer cascade of the cascaded control structure.

Furthermore, the upper limit for the final gas flow F_{nSet} determined in step 176 is the previously determined maximum mean gas flow Fₘₐₓ and the lower limit is the previously determined minimum gas flow Fₘᵢₙ (step 177).

Next, the measured, internal gas flow Fᵢₙₜ is adjusted towards the calculated final gas flow F_{nSet} with the aid of the actuator 122 (step 178). In other words, a zero adjustment is carried out, in accordance with:

F_{nSet}−Fᵢₙₜ→0

so that the nasal gas flow Fₙₐₛₐₗ is adjusted towards the calculated final gas flow F_{nSet}. In this manner, the flow control is carried out as the inner cascade of the cascaded control structure.

Next, the control device 128 generates a suitable control command for the actuator 122 on the basis of step 178 and transfers it to the actuator 122, so that the actuator 22 delivers the conditioned gas into the tubing system (step 179; step d)).

Subsequently, steps 170 to 179 can be carried out several times.

### LIST OF REFERENCE NUMERALS

15 system
16 nasal canula
17 tubing system
18 tubing
19 humidifier
20 medical apparatus
22 actuator
23 tubing system connection
24 digital-to-analogue transducer for 22
28 control device
30 computer system
32 storage unit
33 table
35 pressure controller
36 cascaded control structure
38 internal pressure measuring sensor
39 analogue-to-digital transducer
40 flow controller
41 internal flow measuring sensor
42 analogue-to-digital transducer for 41
45 input and output device
46 display unit
47 touch screen
48 switch device
49 terminal strip
120 medical apparatus
122 actuator
124 digital-to-analogue transducer for 122
128 control device
135 pressure controller
136 cascaded control structure
145 input and output device
140 flow controller
149 terminal strip
150 analogue-to-digital transducer
160 pressure measuring device
70-91 steps of method
170-179 steps of method
P_{nSet} first final nasal gas pressure
Pₙₐₛₐₗ nasal gas pressure
Pₘₑₛ measured pressure value
Pₙ pressure approximation
dP_{sch} first differential pressure approximation
Pᵢₙₜ first internal measured pressure value
Fₘᵢₙ minimum gas flow
Fₙₐₛₐₗ nasal gas flow
Fₘₐₓ maximum mean gas flow
Fᵢₙₜ, max effective, internal, maximum gas flow
Fᵢₙₜ internal gas flow
Fᵢₙₜ, min minimum, internal gas flow
F_{nSet} final nasal gas pressure

What is claimed is:

1. A method for operating an actuator (22; 122) in a medical apparatus (20; 120), wherein the actuator (22; 122) is connected to a tubing system (17), and the medical apparatus has a control device (28; 128) with a pressure controller (35; 135) for controlling a gas pressure, as well as a computer system (30), wherein the method comprises the following steps:
a) providing the tubing system (17) with a nasal cannula (16);
b) specifying a final nasal gas pressure (P_{nSet}) in the pressure controller (35; 135);
c) adjusting a nasal gas pressure (Pₙₐₛₐₗ) at the actuator (22; 122) with the aid of the pressure controller (35; 135), in particular towards the final nasal gas pressure (P_{nSet});
d) delivering a conditioned gas from the actuator (22; 122) into the tubing system (17).

2. The method as claimed in claim 1, characterized in that the pressure control (35; 135) is carried out on the basis of at least one measured nasal pressure value (Pₘₑₛ) or on the basis of at least one pressure approximation (Pₙ), wherein in particular, a variation in a nasal gas flow (Fₙₐₛₐₗ) in the tubing system (17) occurs, which preferably occurs before setting the final nasal gas pressure (P_{nSet}) at the pressure controller (35; 135) (step b)).

3. The method as claimed in claim 2, characterized in that the at least one pressure approximation (Pₙ) is calculated on the basis of at least one internal measured pressure value (Pᵢₙₜ) and on the basis of at least one differential pressure approximation (dP_{sch}) in the tubing system (17).

4. The method as claimed in claim 3, characterized in that the at least one differential pressure approximation (dP_{sch}) in the tubing system (17) is calculated on the basis of an internal gas flow (Fᵢₙₜ), wherein the at least one differential pressure approximation (dP_{sch}) is in particular calculated with the aid of a mathematical function, or preferably, is called up from a table (33) in the computer system (30), or more particularly calculated with the aid of at least one measured nasal pressure value (Pₘₑₛ).

5. The method as claimed in one of claims 1 to 4, characterized in that a minimum gas flow (Fₘᵢₙ) is determined or controlled through the tubing system (17), wherein the minimum gas flow (Fₘᵢₙ) is calculated in the computer system (30) using:
$${F}_{min} = \frac{{V}_{d}}{k * {T}_{e}}$$
wherein V_{d} is the dead space which in particular is calculated with the aid of an ideal body weight of a patient and a Radford constant, and k is a factor between 0.05 and 2, preferably 0.33, and Tₑ is an expiration time.

6. The method as claimed in one of claims 1 to 5, characterized in that a maximum mean gas flow (Fₘₐₓ) is provided through the tubing system (17), wherein the mean, maximum gas flow (Fₘₐₓ) is preferably set to a value between 10 and 200 litres per minute, preferably to a value of 100 litres per minute.

7. The method as claimed in one of claims 1 to 6, characterized in that the nasal gas flow (Fₙₐₛₐₗ) is controlled, wherein the nasal gas flow (Fₙₐₛₐₗ) is preferably controlled as the inner cascade of the nasal gas pressure (Pₙₐₛₐₗ).

8. The method as claimed in one of claims 1 to 7, characterized in that setting the final nasal gas pressure (P_{nSet}) (step b)) is carried out automatically, wherein in particular, this is adjusted on the basis of a measured blood gas value, preferably a carbon dioxide value or an oxygen saturation value or a minimum gas flow (Fₘᵢₙ).

9. A device for carrying out a nasal flow therapy application, comprising an actuator (22; 122), a tubing system connection (23) for connecting a tubing system (17) to the actuator (22; 122) as well as at least one transducer (39), which provides measuring signals from at least one pressure measuring sensor (38), characterized in that a control device (28; 128) with a pressure controller (35; 135) is provided for controlling a nasal gas pressure (Pₙₐₛₐₗ).

10. The device as claimed in claim 9, characterized in that a computer system (30) is provided, wherein the actuator (22; 122) is connected to the computer system (30) and the computer system (30) preferably has a storage unit (32), and the computer system (30) is configured in order to calculate at least one pressure approximation (Pₙ) on the basis of at least one internal measured pressure value (Pᵢₙₜ) and on the basis of at least one differential pressure approximation (dP_{sch}) in the tubing system (17), and preferably the pressure controller (35; 135) is configured to control the nasal gas pressure (Pₙₐₛₐₗ) pertaining to the tubing system (17) on the basis of the at least one pressure approximation (Pₙ).

11. The device as claimed in claim 9 or claim 10, characterized in that a pressure measuring device (160) is provided for detecting at least one measured nasal pressure value (Pₘₑₛ), wherein the pressure controller (35; 135) controls the nasal gas pressure (Pₙₐₛₐₗ) pertaining to the tubing system (17) on the basis of the at least one measured nasal pressure value (Pₘₑₛ), and preferably a flow measuring sensor (41) is provided for measuring an internal gas flow (Fᵢₙₜ) in the medical apparatus (20; 120).

12. The device as claimed in one of claims 9 to 11, characterized in that a humidifier (19) is provided, wherein in particular, the humidifier (19) is disposed on the tubing system (17), and/or a temperature control system is provided for controlling the temperature of the conditioned gas.

13. The device as claimed in one of claims 9 to 12, characterized in that a flow controller (40; 140) is provided for controlling a nasal gas flow (Fₙₐₛₐₗ), and in particular a cascaded control structure (36; 136) is provided, wherein the pressure controller (35; 135) forms an outer cascade for controlling the nasal gas pressure (Pₙₐₛₐₗ) and a flow controller (40; 140) forms an inner cascade for controlling the nasal gas flow (Fₙₐₛₐₗ).

14. The device as claimed in one of claims 9 to 13, characterized in that an oxygen dosing device is provided, and preferably an input and output device (45; 145) is provided, wherein in particular, the input and output device (45; 145) has a display unit (46) and is preferably a touch screen (47).

15. The device as claimed in one of claims 9 to 14, characterized in that a measuring device is provided for measuring blood gas, preferably for measuring carbon dioxide and/or for measuring oxygen.
